SEQUENCE LISTING (41 sequences)

SEQ ID NO: 1 - DNA, 1362 bp, Glycine max
gatcaatcacgatcttctaataagaacaagaaatctttttcgcgatcaatccttttgtcccattcttcaataatcagaaagatccttttcaatcaagtttgaattttttcgtttggaatcaggactcttctactgcatttttatttactttttttttatttcttttcttccatcattccttaactcccacaaggtttggtcctgtagaatctgacccatttcatcattgagcgaaaagtacgaaaaaaatcagatcgatttttcgaccaaaagtactatgtgaaatcctcggttttttcctctttctctatccctatctcgtaggtagagcgtttgaatcaatagagaaccctttcttctgtatctgtatgaatcgatattattacattccaaaattccttcccgatacctcctaaggaaccgaattggatcccaaattgacgggttagtgtgagcttatccatgcggttatgcacccttcgaataggaatccattttctgaaagatcccggctttcgtgcgttggtgggtcttcgagatcctttcgatgacctatgttgtgttgaagggatatctatatgaaaagacagttctatttctattctattagtattttcgattagtattaaattcgttttagttagtgatctcggctcagctagtcctttctttcgtgatgaactgttggcacctgtcttacattttgtctctgtggaccgaggagaaagggagctcagcggcaagaggattgtaacatgagagaagcaaggaggtcaacctttttcaaatatacaacatgggttctggcaatgcaatgtggttggactctcatgtcgatctgaatgaatcatcctttccacggaggtaaatctttgcctgctaggcaagagtatagcaaattacaaattctgtcttggtagggcatgtatttttattactattaaattgaagtagttaatggtggggttaccattatcctttttgtggtaacgaatatgtgttcctaagaaaagcaatttgtccattttttcggggtctcgaaggggcgtggaaacacataagaactcttgaattgaaatggaaaaatagatgtaactccagttacttcggaaatggtaagatctttggcgcaagaacgcaagaggaggggttgatccgtatcatcttgacttggttctgatttctctattttttaataaaatcgagtcgggttcttctcctacccgtatcgaatagaacatgcttagccaaatcttcttcatggaaaacctgctttatttagatcgggaaaatcatatggttttatgaaatcatgtgctattgctcgaatccgtggtcaatcctatttccgatagagcagtt

SEQ ID NO: 2 - DNA, 1763 bp, Glycine max
gacaatggaatccaatttttccataattttcgtatccgtaatagtgtgaaaagaaagcctaactccaagaagttgtttaagaatagtggcgttgagtttcttgaccctttgccttaggattagtcagttctatttctcgatggaggcaagggatataactcagcggtagagtgtcaccttgacgtggtggaagttatcagttcgagcctgattatccctaaacccaatgtaagtttttctatttgtatgccgtgatcgaataataattgagaatggataagaggctcgtgggattacacgaggggtgggggggctatatttctgggagcgaactccagtcgaatatgaagcgcctggatacaagttatgccttggaatggaagagaattccgaatcagctttgtctacgaacaaggaagctataagtaatgcaactaggaatctcatggagagttcgatcctggctcaggatgaacgctggcggcatgccttacacatgcaagtcggacgggaagtggtgtttccagtggcggacgggtgagtaacgcgtaagaacctacccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtgaaggtagaaggcctacgggtcatgaacttcttttcccggagaagaagcaatgacggtatccggggaataagcatcggctaactctgtgccagcagccgcggtaagacagaggatgcaagcgttatccggaatgattgggcgtaaagcgtctgtaggtggctttttaagttcgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcaatgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccaacatttagtttggaaccctgagcagactgccggtgataagccggaggaaggtgaggatgacgtcaagtcatcatgccccttatgccctgggcgacacacgtgctacaatggacgggacaaaggatcgcgatcccgcgagggtgagctaactccaaaaacccgtcctcagttcggattgtaggctgcaactcgcctgcatgaagccggaatcgctagtaatcgccggtcagccatacggcggtg

SEQ ID NO: 3 - DNA, 30 bp, Artificial Sequence
  Synthetic sequence OSSD3
ctaggagctccaccgccgtatggctgaccg

SEQ ID NO: 4 - DNA, 63 bp, Artificial Sequence
  Synthetic sequence OSSD5
gtcgaccatggactagtccaccgcggtggtctagactcgaggacaatggaatccaatttttcc

SEQ ID NO: 5 - DNA, 50 bp, Artificial Sequence
  Synthetic sequence OSSG3
ctctccatgggttaacaagcttaactgctctatcggaaataggattgacc

SEQ ID NO: 6 - DNA, 39 bp, Artificial Sequence
  Synthetic sequence OSSG5
ctagtggtaccgatccaatcacgatcttctaataagaac

SEQ ID NO: 7 - DNA, 40 bp, Artificial Sequence
  Synthetic sequence OSSG310
gaacctccttgcttctctcatgttacaatcctcttgccgc

SEQ ID NO: 8 - DNA, 43 bp, Artificial Sequence
  Synthetic sequence OAAX3
ctcagtactcgagttatttgccgactaccttggtgatctcgcc

SEQ ID NO: 9 - DNA, 34 bp, Artificial Sequence
  Synthetic sequence OAAN5
gaagcttccatggcagaagcggtgatcgccgaag

SEQ ID NO: 10 - DNA, 1327 bp, Artificial sequence
  Synthetic sequence AADA312
gctcccccgccgtcgttcaatgagaatggataagaggctcgtgggattgacgtgagggggcagggatggctatatttctgggagcgaactccgggcgaatacgaagcgcttggatacagttgtagggagggatccatggcagaagcggtgatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatctagagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcg

SEQ ID NO: 11 - DNA, 1330 bp, Artificial sequence
  Synthetic sequence AADA166
gctcccccgccgtcgttcaatgagaatggataagaggctcgtgggattgacgtgagggggcagggatggctatatttctgggagcgaactccgggcgaatacgaagcgcttggatacagttgtagggagggatttatggatcccgaagcggtgatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatctagcgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcg

SEQ ID NO: 12 - DNA, 1746 bp, Artificial sequence
  Synthetic sequence AOA170
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatgggcaagggcgaggaactgttcactggcgtggtcccaatcttaagctccatggaatccctgacgttacaacccatcgcgcgggtcgatggcgccattaatttacctggctccaaaagtgtttcaaaccgtgctttgctcctggcggctttagcttgtggtaaaaccgctctgacgaatctgctggatagcgatgacgtccgccatatgctcaatgccctgagcgcgttggggatcaattacaccctttctgccgatcgcacccgctgtgatatcacgggtaatggcggcgcattacgtgcgccaggcgctctggaactgtttctcggtaatgccggaatcgcgatgcgttcgttagcggcagcgctatgtctggggcaaaatgagatagtgttaaccggcgaaccgcgtatgaaagagcgtccgataggccatctggtcgattcgctgcgtcagggcggggcgaatattgattacctggagcaggaaaactatccgcccctgcgtctgcgcggcggttttaccggcggcgacattgaggttgatggtagcgtttccagccagttcctgaccgctctgctgatgacggcgccgctggcccctaaagacacaattattcgcgttaaaggcgaactggtatcaaaaccttacatcgatatcacgctaaatttaatgaaaacctttggcgtggagatagcgaaccaccactaccaacaatttgtcgtgaagggaggtcaacagtatcactctccaggtcgctatctggtcgagggcgatgcctcgtcagcgtcctattttctcgccgctggggcgataaaaggcggcacggtaaaagtgaccggaattggccgcaaaagtatgcagggcgatattcgttttgccgatgtgctggagaaaatgggcgcgaccattacctggggcgatgattttattgcctgcacgcgcggtgaattgcacgccatagatatggatatgaaccatattccggatgcggcgatgacgattgccaccacggcgctgtttgcgaaaggaaccacgacgttgcgcaatatttataactggcgagtgaaagaaaccgatcgcctgttcgcgatggcgaccgagctacgtaaagtgggcgctgaagtcgaagaagggcacgactatattcgtatcacgccgccggcgaagctccaacacgcggatattggcacgtacaacgaccaccgtatggcgatgtgcttctcactggtcgcactgtccgatacgccagttacgatcctggaccctaaatgtaccgcaaaaacgttccctgattatttcgaacaactggcgcgaatgagtacgcctgcctaatttaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagc

SEQ ID NO: 13 - DNA, 1694 bp, Artificial sequence
  Synthetic sequence AROA319
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatggaatccctgacgttacaacccatcgcgcgggtcgatggcgccattaatttacctggctccaaaagtgtttcaaaccgtgctttgctcctggcggctttagcttgtggtaaaaccgctctgacgaatctgctggatagcgatgacgtccgccatatgctcaatgccctgagcgcgttggggatcaattacaccctttctgccgatcgcacccgctgtgatatcacgggtaatggcggcgcattacgtgcgccaggcgctctggaactgtttctcggtaatgccggaatcgcgatgcgttcgttagcggcagcgctatgtctggggcaaaatgagatagtgttaaccggcgaaccgcgtatgaaagagcgtccgataggccatctggtcgattcgctgcgtcagggcggggcgaatattgattacctggagcaggaaaactatccgcccctgcgtctgcgcggcggttttaccggcggcgacattgaggttgatggtagcgtttccagccagttcctgaccgctctgctgatgacggcgccgctggcccctaaagacacaattattcgcgttaaaggcgaactggtatcaaaaccttacatcgatatcacgctaaatttaatgaaaacctttggcgtggagatagcgaaccaccactaccaacaatttgtcgtgaagggaggtcaacagtatcactctccaggtcgctatctggtcgagggcgatgcctcgtcagcgtcctattttctcgccgctggggcgataaaaggcggcacggtaaaagtgaccggaattggccgcaaaagtatgcagggcgatattcgttttgccgatgtgctggagaaaatgggcgcgaccattacctggggcgatgattttattgcctgcacgcgcggtgaattgcacgccatagatatggatatgaaccatattccggatgcggcgatgacgattgccaccacggcgctgtttgcgaaaggaaccacgacgttgcgcaatatttataactggcgagtgaaagaaaccgatcgcctgttcgcgatggcgaccgagctacgtaaagtgggcgctgaagtcgaagaagggcacgactatattcgtatcacgccgccggcgaagctccaacacgcggatattggcacgtacaacgaccaccgtatggcgatgtgcttctcactggtcgcactgtccgatacgccagttacgatcctggaccctaaatgtaccgcaaaaacgttccctgattatttcgaacaactggcgcgaatgagtacgcctgcctaatttaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctag

SEQ ID NO: 14 - DNA, 553 bp, Artificial sequence
  Synthetic sequence HELIO312
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatggataaattaattggatcttgtgtatggggagctgtaaattatacttctgattgtaatggagaatgtaaaagaagaggatataaaggaggacattgtggatcttttgctaatgtaaattgttggtgtgaaacttaatctagaggaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctag

SEQ ID NO: 15 - DNA, 1487 bp, Artificial sequence
  Synthetic sequence HPPD323
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatggcagatctatacgaaaacccaatgggcctgatgggctttgaattcatcgaattcgcgtcgccgacgccgggtaccctggagccgatcttcgagatcatgggcttcaccaaagtcgcgacccaccgttccaagaacgtgcacctgtaccgccagggcgagatcaacctgatcctcaacaacgagcccaacagcatcgcctcctactttgcggccgaacacggcccgtcggtgtgcggcatggcgttccgcgtgaaggactcgcaaaaggcctacaaccgcgccctggaactcggcgcccagccgatccatattgacaccgggccgatggaattgaacctgccggcgatcaagggcatcggcggcgcgccgttgtacctgatcgaccgtttcggcgaaggcagctcgatctacgacatcgacttcgtgtacctcgaaggtgtggagcgcaatccggtcggtgcaggtctcaaagtcatcgaccacctgacccacaacgtctatcgcggccgcatggtctactgggccaacttctacgagaaattgttcaacttccgtgaagcgcgttacttcgatatcaagggcgagtacaccggcctgacttccaaggccatgagtgcgccggacggcatgatccgcatcccgctgaacgaagagtcgtccaagggcgcggggcagatcgaagagttcctgatgcagttcaacggcgaaggcatccagcacgtggcgttcctcaccgacgacctggtcaagacctgggacgcgttgaagaaaatcggcatgcgcttcatgaccgcgccgccagacacttattacgaaatgctcgaaggccgcctgcctgaccacggcgagccggtggatcaactgcaggcacgcggtatcctgctggacggatcttccgtggaaggcgacaaacgcctgctgctgcagatcttctcggaaaccctgatgggcccggtgttcttcgaattcatccagcgcaagggcgacgatgggtttggcgagggcaacttcaaggcgctgttcgagtccatcgaacgtgaccaggtgcgtcgtggtgtattgaccgccgattaatttaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctag

SEQ ID NO: 16 - DNA, 3929 bp, Artificial sequence
  Synthetic sequence CRYL325
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatgggcaagggcgaggaactgttcactggcgtggtcccaatcttaagctccatggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctacttaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggcgcctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataatttaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctag

SEQ ID NO: 17 - DNA, 3878 bp, Artificial sequence
  Synthetic sequence CRYL327
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctacttaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggcgcctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataatttaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctag

SEQ ID NO: 18 - DNA, 2261 bp, Artificial sequence
  Synthetic sequence CRYS329
gctcccccgccgtcgttcaatgagaatggataagaggctcgtgggattgacgtgagggggcagggatggctatatttctgggagcgaactccgggcgaatactgaagcgcttggatacaagttatccttggaaggaaagacaattccgaatctagaaataattttgtttaactttaagaaggagatatacccatggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgattaatttaaatagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctag

SEQ ID NO: 19 - DNA, 48 bp, Artificial sequence
  Synthetic sequence OTPRRNC5
caattgtcgcgagaattcgctagcggcgccgctcccccgccgtcgttc

SEQ ID NO: 20 - DNA, 59 bp, Artificial sequence
  Synthetic sequence OTPRRNC3
atcgatccgcgggagctcggtaccatgcatcgtctagattcggaattgtctttccttcc

SEQ ID NO: 21 - DNA, 57 bp, Artificial sequence
  Synthetic sequence OG10L5
tatctagaaataattttgtttaactttaagaaggagatatacccatgggcaagggcg

SEQ ID NO: 22 - DNA, 67 bp, Artificial sequence
  Synthetic sequence OPGFP3
ggatgcattgcttaagattgggaccacgccagtgaacagttcctcgcccttgcccatgggtatatct

SEQ ID NO: 23 - DNA, 36 bp, Artificial sequence
  Synthetic sequence OAROADB5
gccttaagctccatggaatccctgacgttacaaccc

SEQ ID NO: 24 - DNA, 38 bp, Artificial sequence
  Synthetic sequence ORAOADB3
gcgatgcataatttaaattaggcaggcgtactcattcg

SEQ ID NO: 25 - DNA, 40 bp, Artificial sequence
  Synthetic sequence OSMC5
gaaagcttcggaccgtagtttaaacaggcccatatggcct

SEQ ID NO: 26 - DNA, 39 bp, Artificial sequence
  Synthetic sequence OSMC3
smcgactcgagttaattaatcggcgcgccaggccatatg

SEQ ID NO: 27 - DNA, 48 bp, Artificial sequence
  Synthetic sequenceOSMC51
gagcggccgcctcgagcggaccgtagtttaaacaggcccatatggcct

SEQ ID NO: 28 - DNA, 36 bp, Artificial sequence
  Synthetic sequence OSMC31
gaaagcttttaattaatcggcgcgccaggccatatg

SEQ ID NO: 29 - DNA, 42 bp, Artificial sequence
  Synthetic sequence OHPPD5
gccttaagctccatggcagatctatacgaaaacccaatgggc

SEQ ID NO: 30 - DNA, 43 bp, Artificial sequence
  Synthetic sequence OHPPD3
gccatttaaattaatcggcggtcaatacaccacgacgcacctg

SEQ ID NO: 31 - DNA, 37 bp, Artificial sequence
  Synthetic sequence OCRYWT5
gccttaagctccatggataacaatccgaacatcaatg

SEQ ID NO: 32 - DNA, 47 bp, Artificial sequence
  Synthetic sequence OCRYWTL3
gccatttaaattattcctccataagaagtaattccacgctgtccacg

SEQ ID NO: 33 - DNA, 49 bp, Artificial sequence
  Synthetic sequence OCRYWTC3
gccatttaaattaatcatattctgcctcaaaggttacttctgccggaac

SEQ ID NO: 34 - DNA, 23 bp, Artificial sequence
  Synthetic sequence P1
cgtatcgaatagaacatgcttag

SEQ ID NO: 35 - DNA, 22 bp, Artificial sequence
  Synthetic sequence P2
acaccatggataaattaattgg

SEQ ID NO: 36 - DNA, 25 bp, Artificial sequence
  Synthetic sequence P3
cctctagattaagtttcacaccaac

SEQ ID NO: 37 - DNA, 24 bp, Artificial sequence
  Synthetic sequence P4
cgtcatacttgaagctagacaggc

SEQ ID NO: 38 - DNA, 43 bp, Artificial sequence; identical to SEQ ID NO: 8
  Synthetic sequence P5

SEQ ID NO: 39 - DNA, 24 bp, Artificial sequence
  Synthetic sequence P6
gttaaggtaacgacttcggcatgg

SEQ ID NO: 40 - DNA, 24 bp, Artificial sequence
  Synthetic sequence P7
catgggttctggcaatgcaatgtg

SEQ ID NO: 41 - DNA, 26 bp, Artificial sequence
  Synthetic sequence P8
caggatcgaactctccatgagattcc

(19) United States
(12) Patent Application Publication
(10) Pub. No.: US 2007/0039075 A1
(43) Pub. Date: Feb. 15, 2007

(54) Fertile transplastomic leguminous plants

(75) Inventors: Ghislaine Tissot, Vancia (FR); Nathalie Dufourmantel, Caluire Et Cuire (FR); Frederic Garcon, Lyon (FR); Jean-Marc Ferullo, Lyon (FR); Bernard Pelissier, Saint Didier Au Mont D'Or (FR)

Correspondence Address: CONNOLLY BOVE LODGE & HUTZ, LLP, P O BOX 2207, WILMINGTON, DE, 19899, US

(73) Assignee: Bayer CropScience S.A., Lyon (FR)

(21) Appl. No.: 10/538,130

(22) PCT Filed: Dec. 8, 2003
(86) PCT No.: PCT/EP03/15007

§ 371 Date: Jun. 29, 2006

(30) Foreign Application Priority Data
Dec. 6, 2002 (FR) 0215490

(51) Int. Cl. A01H 5/00; C12N 15/82

(52) U.S. Cl. 800/312; 800/313; 435/468

(57) ABSTRACT

The invention relates to the transformation of plastids from plants, and more precisely to the production of fertile transplastomic leguminous plants, in particular of fertile transplastomic soybean.

The invention relates to the transformation of plastids from plants, and more precisely to the production of fertile transplastomic leguminous plants, in particular of fertile transplastomic soybean.

### STATE OF THE ART

In plants, the genetic information is distributed into three cell compartments: the nucleus, the mitochondria and the plastids. Each of these compartments carries its own genome. For some years, plastids of higher plants have been an attractive target for genetic manipulations. Plastids from plants (chloroplasts, site of photosynthesis, starch-accumulating amyloplasts, elaioplasts, etioplasts, carotenoid-accumulating chromoplasts, etc.) are major centers of biosynthesis which, besides photosynthesis, are responsible for the production of industrially important compounds such as aminoacids, carbohydrates, fatty acids and pigments. Plastids are derived from a common undifferentiated precursor, the proplastid, and therefore, in a given plant species, have the same genetic content.

The plastid genome, or plastome, of higher plants consists of a double-stranded circular DNA molecule of 120-160 kilobases, carrying a large repeated and inverted sequence (approximately 25 kb). A notable characteristic of the plastid genome lies in the presence of many identical copies of this genome in all the cells and all the plastid types. Depending on the stage of development, a tobacco leaf cell may contain up to 10 000 plastome copies. It is therefore possible to manipulate plant cells containing up to 20 000 copies of a gene of interest, which can potentially result in a high level of heterologous gene expression.

The transformation of plastid genomes from plants offers an enormous potential for plant biotechnology and many very attractive advantages compared to conventional transformation of the nuclear genome. The first advantage lies in the very mechanism of plastid transformation. Specifically, the integration of a transgene into the plastome takes place by a phenomenon of double homologous recombination. This process makes it possible to precisely target the region of the plastome at which integration of the gene of interest is desired, in particular using plastid sequences positioned on either side of the transgene on the transformation vector. This precise targeting avoids the “position” effect commonly observed in nuclear transformation events.

The second advantage lies in the high number of transgene copies per plastid. The plant cells can be manipulated so as to contain up to 20 000 copies of a gene of interest. This characteristic allows high levels of transgene expression which may result in an accumulation of recombinant proteins ranging up to 40% of total soluble cell proteins (De Cosa et al., 2001, Nat. Biotechnol. 19, 71-74).

The prokaryotic nature of the plastid constitutes another attribute, in particular by allowing the expression of genes organized in operons and the efficient translation of polycistronic mRNAs. This particularity facilitates the coordinated functioning of several transgenes, while at the same time limiting the number of transformation steps and the need to use multiple selection markers (Daniell, 1998, Nat. Biotechnol. 16, 345-8; De Cosa et al., 2001, Nat. Biotechnol. 19, 71-74).

Another advantage of plastid transformation compared to nuclear transformation lies in the control of transgene dispersion in the environment. In many angiosperms, the plastids have a strict maternal heredity, and the plastid DNA is not transmitted via the pollen. This particularity therefore greatly limits the risk of dispersion of the transgene in the environment, and its potential propagation to neighboring plants.

Many applications of plastid transformation have made it possible to confirm the advantages of this technology over nuclear transformation. Thus, overexpression, from the tobacco plastome, of genes for tolerance to herbicides such as glyphosate (Daniell, 1998, Nat. Biotechnol. 16, 345-8; WO99/10513; Ye et al., 2000; WO 01/04331, WO 01/04327), or phos-phinothricin (Basta) (Lutz et al., 2001, Physiol. Plant 125, 1585-1590), confers excellent tolerance to these herbicides. Other applications have led to the production of transplastomic plants which are tolerant to insects or which overproduce therapeutic proteins (McBride et al., 1995; U.S. Pat. No. 5,451,513; Staub et al., 2000, Nat. Biotech. 18, 333-338).

To obtain plastid transformation, the transforming DNA must cross the cell wall, the plasma membrane and the double membrane of the organelle before reaching the stroma. In this respect, the most commonly used technique for transforming the plastid genome is that of particle bombardment (Svab and Maliga, 1993, Proc. Natl. Acad. Sci. USA, Feb 1, 90(3): 913-7).

Currently, in higher plants, stable transformation of plastids is commonly carried out only in tobacco, Nicotiana tabacum (Svab and Maliga, 1990 Proc. Natl. Acad. Sci. USA 87, 8526-8530; Svab and Maliga, 1993, Proc. Natl. Acad. Sci. USA, Feb 1, 90(3): 913-7). Although this technique has demonstrated its effectiveness in tobacco, its transposition to large crop plant species appears to come up against technical obstacles. One of these obstacles may be not a difficulty in transformation, but probably a limitation in the systems for in vitro culturing of tissues currently available and in the methods of transformation and of regeneration of transplastomic plants. Some recent progress has, however, been achieved with the transformation of plastids from rice (Khan M.S. and Maliga, 1999, Nat. Biotechnol. 17, 910-915), from Arabidopsis thaliana (Sikdar et al., 1998, Plant Cell Reports 18:20-24), from potato (Sidorov et al., 1999, Plant J. 19(2): 209-216), from Brassica napus (Chaudhuri et al., 1999) and from tomato (Ruf et al., 2001, Nat. Biotechnol. 19, 870-875).

Recently, Zhang et al. (2001, J. Plant Biotechnol. 3, 39-44) have described a technique for transforming plastids from a soybean cell suspension at very low frequency. However, this technique yields tissues incapable of regenerating plants. To the inventors' knowledge, no fertile transplastomic leguminous plant, and more particularly no fertile transplastomic soybean plant, has been obtained to date.

A large number of crop species belong to the leguminous plant family, in particular protein-yielding plants such as pea, fababean, bean, chickpea, lentils, oil-yielding plants such as soybean and groundnut, and forage such as alfalfa or clover. A fundamental property of leguminous plants, which is greatly responsible for their agronomic value, is their high protein content. This property makes them plants of choice for overexpressing proteins of interest.

Soybean, essentially grown in North and Latin America, and also in China, is exported in the main to Europe. Over the last few years, characteristics of resistance to a herbicide or to insect pests have been introduced into the nuclear genome of soybean. These genetic manipulations in the nuclear genome of soybean have been accomplished by virtue of the particle bombardment technique. Many genotypes have thus been produced which exhibit an increase in tolerance to herbicides (Roundup Ready Soybean, Pagette et al. 1995, Crop Sci. 35, 1451-1461) or to insect pests (Stewart et al., 1996, Plant Physiol. 112: 121-129), or an improvement in characteristics of quality, such as fatty acids, phytate, aminoacids (Soy 2000, 8^{th} biennial Conference of the cellular and Molecular biology of the soybean, Lexington, Ky.).

In this context, and in view of the technical advantages of plastid transformation mentioned above, it is becoming crucial to develop a reliable technique for transforming and regenerating fertile transplastomic leguminous plants, in particular soybean. Thus, the inventors have developed a method for high frequency transformation of soybean plastomes leading to fertile plants. This method can readily be adapted to the transformation of other leguminous plants of agronomic interest.

### DESCRIPTION

The present invention relates to a fertile transplastomic leguminous plant.

According to the present invention, the term “leguminous plant” is intended to mean a plant of the Fabaceae family. Preferred leguminous plants according to the invention are the leguminous plants of agronomic interest, such as pea (Pisum sativum), broadbean (Vicia faba major), faba bean (Vicia faba minor), lentils (Lens culinaris), bean (Phaseolus vulgaris), chickpea (Cicer arietinum), soybean (Glycine max), groundnut (Arachis hypogea), alfalfa (Medicago sativa) or clover (Trifolium sp.)

According to a preferred embodiment of the invention, the fertile transplastomic leguminous plant is soybean, Glycine max.

According to the invention, the term “transplastomic” is intended to mean plants which have stably integrated into their plastome at least one expression cassette which is functional in plastids. The plastome consists of the genome of the cellular organelles other than the nucleus and the mitochondria. An expression cassette according to the invention comprises, among other elements, at least one promoter which is functional in plastids of plant cells, a sequence encoding a protein of interest and a terminator which is functional in plastids of plant cells. Said expression cassette may contain genetic elements originating from the transformed plant or from any other organism. Also, the expression cassette may contain more than one sequence encoding a protein of interest, like for example in the case of operons.

Preferably, the transplastomic leguminous plants according to the invention are in the homoplasmic state. The homoplasmic state corresponds to a state according to which all the cells contain a population of identical plastomes. According to the invention, transplastomic plants are in the homoplasmic state when all their cells contain only copies of transformed plastomes, and no longer any copies of nontransformed plastomes. This state is generally obtained by selection of the copies of plastomes which have integrated the expression cassette, in particular by means of combining said expression cassette with a gene encoding a selection marker. The plastomes which have not integrated the selection marker are then eliminated when the transformed tissues are brought into contact with the corresponding selection agent.

According to the invention, the transplastomic leguminous plants are fertile. A fertile plant is a plant capable of producing a viable lineage by virtue of a sexual reproductive cycle. In particular, a fertile plant according to the invention is a transplastomic plant capable of transmitting the expression cassette integrated into its plastome into its descendants.

The invention also comprises transformation vectors suitable for plastid transformation. The expression “vector suitable for plastid transformation” is intended to mean a vector capable of stably integrating the expression cassette(s) which it contains into the plastome of plant cells. Advantageously, a vector suitable for plastid transformation according to the invention is a vector comprising at least two sequences homologous with a zone of the plastome of the leguminous plant to be transformed, said homologous sequences bordering at least one expression cassette. According to a preferred embodiment, said homologous sequences border, in addition to an expression cassette encoding one or more proteins of interest, at least one other expression cassette encoding a selection marker. With such vectors, integration of the expression cassette(s) into the plastome is carried out by double homologous recombination of the two sequences homologous with a zone of the plastome of the leguminous plant to be transformed, present on the vector, with the corresponding sequences in the plastome of the leguminous plant to be transformed. Advantageously, the two sequences homologous with a zone of the plastome of the leguminous plant to be transformed allow integration of the expression cassette(s) into an intergenic zone of the plastid genome without interrupting the integrity or the function of the plastid genes. Preferably, this zone corresponds to the region of the ribosomal RNA operon of the plastome.

According to a particular embodiment of the invention, the sequences homologous with a zone of the plastome or the leguminous plant to be transformed correspond to sequences exhibiting 80% identity with the corresponding sequences in the plastome of the leguminous plant to be transformed, preferably 90% identity, preferably 95%, and preferably 99% identity. According to a preferred embodiment of the invention, the sequences homologous with a zone of the plastome of the leguminous plant to be transformed correspond to sequences exhibiting 100% identity with the corresponding sequences in the plastome of the leguminous plant to be transformed.

The invention therefore relates to a vector suitable for plastid transformation, characterized in that the two sequences homologous with a zone of the plastome of the leguminous plant to be transformed correspond to sequences which allow integration of the expression cassette into a plastome intergenic region. According to a preferred embodiment, said zone corresponds to the region of the ribosomal RNA operon of the plastome.

The invention also comprises a fertile transplastomic leguminous plant, characterized in that it comprises at least one expression cassette inserted into a plastome intergenic region. According to a preferred embodiment, said intergenic region is selected from the region of the ribosomal RNA operon of the plastome.

According to a particular embodiment of the invention, one of the two homologous sequences comprises the genes, or a portion thereof, encoding the 16S ribosomal RNA (16SrRNA) and the Valine transfer RNA (trnV), and the other homologous sequence comprises the intergenic region, or a portion thereof, located between the trnV gene and the rps12/7 operon. The invention therefore relates to a vector suitable for plastid transformation, characterized in that one of the two homologous sequences comprises the genes encoding the 16S ribosomal RNA (16SrRNA) and the Valine transfer RNA (trnV), and in that the other homologous sequence comprises the intergenic region located between the trnV gene and the rps12/7 operon.

The invention therefore also comprises a fertile transplastomic leguminous plant, characterized in that it comprises at least one expression cassette inserted into a plastome intergenic region, said plastome intergenic region being located between the trnV gene and the rps12/7 operon.

According to a preferred embodiment of the invention, the leguminous plant to be transformed is soybean. According to this embodiment, the sequence comprising the genes encoding the 16S ribosomal RNA (16SrRNA) and the Valine transfer RNA (TrnV) corresponds to the sequence represented by the identifier SEQ ID No. 1, and the sequence comprising the intergenic region located between the TrnV gene and the rps12/7 operon corresponds to the sequence represented by the identifier SEQ ID No. 2. The invention therefore relates to a vector suitable for plastid transformation, characterized in that the homologous sequence comprising the genes encoding the 16S ribosomal RNA (16SrRNA) and the Valine transfer RNA (TrnV) is represented by the sequence identifier SEQ ID No. 1, and in that the homologous sequence comprising the intergenic region located between the TrnV gene and the rps12/7 operon is represented by the sequence identifier SEQ ID No. 2.

According to a particular embodiment, the invention therefore comprises a fertile transplastomic soybean plant, characterized in that it comprises at least one expression cassette inserted into a plastome intergenic region, said expression cassette being inserted between the soybean plastome sequences corresponding to the identifiers SEQ ID No. 1 and SEQ ID No. 2.

According to a preferred embodiment of the invention, the homologous sequence comprising the genes encoding the 16S ribosomal RNA (16SrRNA) and the Valine transfer RNA (TrnV) is positioned 5′ of the expression cassette, and the homologous sequence comprising the intergenic region located between the TrnV gene and the rps12/7 operon is positioned 3′ of the expression cassette. In another embodiment, the two homologous sequences can be positioned in the reversed position with respect to the expression cassette.

The transformation vectors suitable for plastid transformation according to the invention comprise at least one expression cassette. An expression cassette according to the invention comprises, functionally linked to one another, at least one promoter which is functional in plastids from plant cells, a sequence encoding a protein of interest and a terminator which is functional in plastids from plant cells. The expression “functionally linked to one another” means that said elements of the expression cassette are linked to one another in such a way that their function is coordinated and allows expression of the coding sequence. By way of example, a promoter is functionally linked to a coding sequence when it is capable of ensuring expression of said coding sequence. The construction of an expression cassette according to the invention and the assembly of its various elements can be carried out using techniques well known to those skilled in the art, in particular those described in Sambrook et al. (1989, Molecular Cloning: A Laboratory Manual, Nolan C. ed., New York: Cold Spring Harbor Laboratory Press). The choice of the regulatory elements making up the expression cassette depends essentially on the plant and on the type of plastid in which they must function, and those skilled in the art are capable of selecting regulatory elements which are functional in a given plant.

Among promoters which are functional in plastids from plant cells, mention may be made, by way of example, of the promoter of the psbA gene, encoding the D1 protein of PSII (Staub et al., 1993, EMBO Journal 12(2): 601-606), or the constitutive promoter of the ribosomal RNA operon, Prrn (Staub et al., 1992, Plant Cell 4: 39-45). In general, any promoter derived from a plant plastome gene will be suitable, and those skilled in the art will be able to make the appropriate choice from the various available promoters so as to obtain a desired mode of expression (constitutive or inducible). A preferred promoter according to the invention comprises the tobacco Prrn promoter combined with a 5′ portion of the 5′ untranslated region of the tobacco rbcL gene (Svab and Maliga, 1993, Proc. Natl. Acad. Sci. 90: 913-917).

Among terminators which are functional in plastids from plant cells, mention may be made, by way of example, of the terminator of the tobacco psbA gene (Shinozaki et al., 1986, EMBO J. 5: 2043-2049; Staub et al., 1993). In general, any terminator derived from a plant plastome gene will be suitable, and those skilled in the art will be able to make the appropriate choice from the various available terminators.

Advantageously, the vector used in the present invention may contain, in addition to an expression cassette comprising a sequence encoding a protein of interest, at least one other expression cassette comprising a sequence encoding a selection marker. The selection marker makes it possible to select the plastids and the cells which have been effectively transformed, i.e. which have incorporated the expression cassette(s) into their plastome. It also makes it possible to obtain fertile transplastomic plastids in the homoplasmic state. Among the useable sequences encoding selection markers, mention may be made of those of the genes for resistance to antibiotics, such as, for example, that of the aadA gene encoding an aminoglycoside 3″-adenyltransferase, which confers resistance to spectinomycin and to streptomycin (Svab et al., 1993; Staub et al., 1993), or that of the hygromycin phosphotransferase gene (Gritz et al., 1983, Gene 25: 179-188), but also those of the genes for tolerance to herbicides, such as the bar gene (White et al., 1990, Nucleic Acid Res. 18(4):1062) for tolerance to bialaphos, the EPSPS gene (U.S. Pat. No. 5,188,642) for tolerance to glyphosate or alternatively the HPPD gene (WO 96/38567) for tolerance to isoxazoles. Use may also be made of the sequences of reporter genes encoding readily identifiable enzymes, such as the GUS enzyme, or sequences of genes encoding pigments or enzymes which regulate the production of pigments in the transformed cells. Such genes are in particular described in patent applications WO 91/02071, WO 95/06128, WO 96/38567 and WO 97/04103.

According to a preferred embodiment of the invention, the gene encoding a selection marker is the aadA gene encoding an aminoglycoside 3″-adenyltransferase, which confers on the transformed cells and plastids resistance to spectinomycin and to streptomycin (Svab et al., 1993; Staub et al., 1993).

The invention also relates to a method for obtaining fertile transplastomic leguminous plants. This method comprises the steps of:

(a) Transforming embryogenic tissues obtained from immature embryos of leguminous plants with a vector suitable for plant transformation,

(b) selecting the transformed tissues,

(c) regenerating fertile transplastomic plants from the transformed tissues.

To implement the method according to the invention, the transformation step (a) should be carried out on embryogenic tissues obtained from immature embryos of leguminous plants. Preferably, the embryogenic tissues are calli or any other tissue containing cells which have conserved a totipotent state.

The embryogenic tissues can be transformed by any method of direct (naked DNA) or indirect transformation of plant cells. Among the methods of transformation which can be used to obtain transplastomic plants according to the invention, one of them consists in bringing the cells or tissues of the plants to be transformed into contact with polyethylene glycol (PEG) and the transformation vector (Chang and Cohen, 1979, Mol. Gen. Genet. 168(1), 111-115; Mercenier and Chassy, 1988, Biochimie 70(4), 503-517). Electroporation is another method, which consists in subjecting the cells or tissues to be transformed and the vectors to an electric field (Andreason and Evans, 1988, Biotechniques 6(7), 650-660; Shigekawa and Dower, 1989, Aust. J. Biotechnol. 3(1), 56-62). Another method consists in directly injecting the vectors into the cells or the tissues by microinjection (Gordon and Ruddle, 1985, Gene 33(2), 121-136). Plastome transformation may also be carried out using bacteria of the genus Agrobacterium, preferably by infection of the cells or tissues of said plants with A. tumefaciens (Knopf, 1979, Subcell. Biochem. 6, 143-173; Shaw et al., 1983, Gene 23(3): 315-330) or A. rhizogenes (Bevan and Chilton, 1982, Annu. Rev. Genet. 16: 357-384; Tepfer and Casse-Delbart, 1987, Microbiol. Sci. 4(1), 24-28). Preferably, the transformation of plant cells or tissues with Agrobacterium tumefaciens is carried out according to the protocol described by Ishida et al. (1996, Nat. Biotechnol. 14(6), 745-750). For plastome transformation, the Agrobacterium strain used should be engineered in such a way as to specifically direct its T-DNA into plastids.

According to a preferred embodiment of the method according to the invention, the “particle bombardment” method will be used. It consists in bombarding the embryogenic tissues with particles, preferably made of gold or tungsten, onto which are adsorbed the vectors according to the invention (Bruce et al., 1989, Proc. Natl. Acad. Sci. USA 86(24), 9692-9696; Finer et al., 1992, Plant Cell Rep. 11, 232-238; Klein et al., 1992, Biotechnology 10(3), 286-291; U.S. Pat. No. 4,945,050).

According to the present method for obtaining fertile transplastomic leguminous plants, the embryogenic tissues are transformed with a vector suitable for plastid transformation, as described in the present invention.

During the step (a) of transforming the embryogenic tissues, not all the tissues subjected to the transformation technique integrate the vector. The step (b) of selecting the transplastomic transformed tissues is carried out by bringing the tissues subjected to the transformation step (a) into contact with the selection agent corresponding to the selection marker gene used. During this phase, only the cells which have integrated the selection marker gene will survive in contact with the selection agent and form green calli. The period of time for which the tissues are brought into contact with the selection agent depends on the selection marker and agent used, and can be readily determined by those skilled in the art. Preferably, this period of time corresponds to a period ranging up to the formation of said green calli from the transformed tissues.

The step (c) of regenerating fertile transplastomic plants from the transformed tissues is carried out by inducing embryo formation from the transplastomic tissues selected in step (b). The induction of embryo formation is generally carried out by bringing said tissues into contact with a suitable embryogenesis medium. Such media are known to those skilled in the art. A preferred medium according to the invention is the medium described in Finer and McMullen (1991).

Once induced, the embryos formed are placed in a suitable medium in order to germinate. Preferably, the medium suitable for germination is an agar medium comprising the nutritive elements required for germination. The young plantlets formed are then planted in a substrate suitable for plant growth. A preferred substrate is earth, or an earth-based mixture.

The invention also comprises parts of the fertile transplastomic leguminous plants and the descendants of these plants. The term “parts” is intended to mean any organ of these plants, whether it is aerial or subterranean. The aerial organs are the stems, the leaves and the flowers comprising the male and female reproductive organs. The subterranean organs are mainly the roots, but they may also be tubers. The term “descendants” is intended to mean mainly the seeds containing the embryos derived from the reproduction of these plants with one another. By extension, the term “descendants” applies to all the seeds formed at each new generation derived from crosses in which at least one of the parents is a transformed plant according to the invention. Descendants may also be obtained by vegetative multiplication of said transformed plants. The seeds according to the invention may be coated with an agrochemical composition comprising at least one active product having an activity selected from fungicidal, herbicidal, insecticidal, nematicidal, bactericidal or virucidal activities.

Among the sequences encoding a protein of interest which can be integrated into the transplastomic leguminous plants according to the invention, mention may be made of the coding sequences of genes encoding an enzyme for resistance to a herbicide, such as, for example, the bar gene encoding the PAT enzyme (White et al., NAR 18: 1062, 1990) which confers tolerance to bialaphos, the gene encoding an EPSPS enzyme (WO 97/04103) which confers tolerance to glyphosate, or the gene encoding an HPPD enzyme (WO 96/38567) which confers tolerance to isoxazoles. Mention may also be made of a gene encoding an insecticidal toxin, for example a gene encoding a δ-endotoxin of the bacterium Bacillus thuringiensis (WO 98/40490). It is also possible to introduce into these plants genes for resistance to diseases, for example a gene encoding the oxalate oxydase enzyme as described in patent application EP 0 531 498 or U.S. Pat. No. 5,866,778, or a gene encoding another antibacterial and/or antifungal peptide, such as those described in patent applications WO 97/30082, WO 99/24594, WO 99/02717, WO 99/53053 and WO 99/91089. It is also possible to introduce genes encoding plant agronomic characteristics, in particular a gene encoding a delta-6 desaturase enzyme as described in U.S. Pat. Nos. 5,552,306 and 5,614,313 and patent applications WO 98/46763 and WO 98/46764, or a gene encoding a serine acetyltransferase (SAT) enzyme as described in patent applications WO 00/01833 and WO 00/36127.

According to a particular embodiment of the invention, the transplastomic leguminous plants according to the invention may be transformed with an expression cassette encoding a protein of pharmaceutical or veterinary interest. By way of example, such a protein may be an anticoagulant (serum protease, hirudin), an interferon or human serum albumin. The proteins produced by the plants according to the invention may also be antibodies, or proteins used as a basis for vaccines.

The examples below make it possible to illustrate the present invention without, however, limiting the scope thereof.

### EXAMPLES

### Example 1

### Construction of a Vector Suitable for Soybean Plastid Transformation

The plasmid pCLT312 contains a heterologous expression cassette, AADA-312, bordered by two soybean plastid DNA fragments, RHRR (Right Homologous Recombination Region) and LHRR (Left Homologous Recombination Region), which allow targeted integration into the region of the ribosomal RNA operon of the soybean plastid. This insertion region is different from that used by Zhang et al. (2001). The RHRR region contains the genes encoding the 16SrRNA (under the control of the ribosomal RNA operon promoter, denoted Prrn) and TrnV (SEQ ID No. 1). The LHRR region contains the intergenic region between the TrnV gene and the rps12/7 operon (SEQ ID No. 2). No plastid gene is interrupted after homologous recombination with these sequences.

The expression cassette of the vector pCLT312 (AADA-312, SEQ ID NO: 10) contains a chimeric gene made up, from 5′ to 3′, of the “short” promoter of the tobacco ribosomal RNA operon (PrrnC, nucleotides 102,564 to 102,715 of the Nicotiana tabacum plastome; Shinozaki et al., 1986), a 5′rbcL portion of the 5′ untranslated region of the tobacco rbcL gene (nucleotides 57 569 to 57 584 of the Nicotiana tabacum plastome; Shinozaki et al., 1986), the coding sequence of the aada gene and the tobacco 3′psbA terminator (nucleotides 533 to 146 of the N. tabacum plastome; Shinozaki et al., 1986). The aadA gene product, an aminoglycoside 3″-adenyltransferase, confers resistance to spectinomycin and to streptomycin on the transformed plants at the level of their plastid genome (Svab et al., 1993; Staub et al., 1993).

The vector pCLT312 was obtained as described below.

The two soybean plastid DNA fragments (constituting the homologous recombination regions RHRR and LHRR) were amplified by PCR from total DNA of Glycine max (cv. Jack) (PWO DNA polymerase, Stratagene). The RHRR region was obtained using the olignucleotides OSSD5 (SEQ ID No. 4) and OSSD3 (SEQ ID No. 3). Annealing (at a temperature of 60° C.) of this pair of primers brought about amplification of a 1 800 bp fragment. In addition, the sequence of these primers generates 5′ and 3′ restriction sites which allow subsequent cloning. The LHRR region was amplified using the primers OSSG5 (SEQ ID No. 6) and OSSG3 (SEQ ID No. 5), designed so as to insert 5′ and 3′ restriction sites. During PCR reaction cycles, the annealing temperature applied is 60° C. The approximately 1 400 bp PCR product obtained is greater in size than that expected (1 180 bp), determined according to the soybean plastome sequence published in GeneBank (X07675). Sequencing of the PCR fragments of these two regions shows the presence of a 217 bp insertion into the LHRR region. This inserted region, according to the analyzed sequence, contains no ORF and is found to be an intergenic region.

After purification on agarose gel, these two PCR fragments, RHRR and LHRR, were cloned into the vector pPCRscript (Strategene) so to give the vectors pCLT309 and pCLT308, respectively. The LHRR region excised from the vector pCLT309 by KpnI digestion was cloned into pCLT308 digested beforehand with this enzyme. A tobacco plastid heterologous expression cassette was then cloned into the vector pCLT300 obtained, using the XhoI and HindIII enzymes, to give the vector pCLT311. This cassette contains a chimeric gene made up, from 5′ to 3′, of the “short” promoter PrrnC of the tobacco ribosomal RNA operon, a 5′rbcL portion of the 5′ untranslated region of the tobacco rbcL gene, the coding sequence of a gene of interest and the tobacco 3′psbA terminator. The gene of interest present in pCLT311 was excised by digestion with the NcoI and XbaI enzymes, and then replaced with the aadA gene released by these same enzymes from the plasmid pCLT115. The plastid transformation vector obtained is called pCLT312.

### Example 2

### Transformation of Soybean Plastid Genomes by Bombardment

The technique used for soybean transformation is particle bombardment. It is applied to embryogenic tissues of soybean. Embryogenic tissues of Glycine max (cv. Jack) were obtained (prepared under sterile conditions) in two phases: an induction phase and a multiplication phase.

Soybean pods are harvested in a greenhouse when the embryos are still immature (maximum of 3 mm in length). They are decontaminated with dilute bleach and rinsed with sterile water. The pods are opened under a hood, under sterile conditions, and the embryos are recovered. The two cotyledons are separated and placed external face down on a D40 agar induction medium. The D40 medium is a Murashige and Skoog medium described in Murashige and Skoog (1962, A revised medium for rapid growth and bioassays with tobacco tissue cultures. Physiol. Plant. 15: 473-479). It comprises (in mg/l): NH₄HO₃: 1650, H₃BO₃: 6.2; CaCl₂.2H₂O: 332.2; CoCl₂.6H₂O: 0.025; CuSO₄.5H₂O: 0.025; Na₂EDTA: 37.26; FeSO₄.7H₂O: 27.8; MnSO₄.7H₂O: 16.9; Na₂MoO₄.2H₂O: 0.25; KI: 0.83; KNO₃: 1990; KH₂PO₄: 170; ZnSO₄.7H₂O: 8.6; Gamborg's B5 vitamin (Gamborg, Miller and Ojima, 1968, Nutrient requirements of suspension cultures of soybean root cells. Exp. Cell Res. 50: 151-158, made up of (in mg/l): myoinositol: 100; nicotinic acid: 1; pyridoxine-HCl: 1; thiamine-HCl: 10), and also 40 mg/l of 2,4-D; 6% saccharose; and 0.3% gelrite, pH 7.0.

This medium is rich in sugar and in 2,4-D, substances which are necessary for the induction of somatic embryos. The embryos are left on this medium for 3 weeks at 24° C., with a given luminosity and photoperiod (16 hours of day and 8 hours of night).

The somatic embryos which have developed at the surface of the cotyledons are recovered and then plated out on D20 medium, which comprises essentially the same elements as the D40 medium, with the exception of the concentration of 2,4-D, which is 20 mg/l, and the concentration saccharose, which is decreased from 60 g/l to 30 g/l, at pH 5.7. This amplification phase lasts 2 weeks on the D20 medium at 28° C.

The embryos are then regularly subcultured on an FNL medium derived from that described by Samoylov et al. (1998). The modified FNL medium comprises (in mg/l): Na₂EDTA: 37.24; FeSO₄, 7H₂O: 27.84; MgSO₄, 7H₂O: 370; MnSO₄, H₂O: 16.9; ZnSO₄, H₂O: 8.6; CuSO₄, 7H₂O: 0.025; CaCl₂, 2H₂O: 440; KI: 0.83; CoCl₂, 6H₂O: 0.025; KH₂PO₄; 170; H₃BO₃: 6.2; Na₂MoO₄, 2H₂O: 0.25; myoinositol: 100; nicotinic acid: 1; pyridoxine-HCl: 1; thiamine-HCl: 10; (NH₄)2SO₄: 460; KNO₃: 2820; asparagine: 670; 1% sucrose; 2,4-D: 10; 0.3% gelrite; pH 5.7. This medium, which is less rich in sugar and 2,4-D, makes it possible to obtain calli suitable for very high frequency transformation in 3 or 4 rounds of subculturing carried out approximately every 15 days.

For the soybean plastid transformation by bombardment, the “FNL” soybean embryogenic tissues are placed at 4° C. for 16 to 20 h. These calli are then placed in a gridded metal capsule and then bombarded on both their faces (front and back) using a “PIG” (Particule Inflow Gun) as described in Finer et al. (1992, Plant Cell Rep. 11, 232-238). Gold microparticles (particles 0.6 μm in diameter) are complexed with the DNA (vector pCLT312, 5 μg/shot) in the presence of CaCl₂ (0.8 to 1 M) and spermidine (14 to 16 mM) according to the methods described in the literature (Russell et al., 1992). The bombarded soybean embryogenic calli are then cut up into small pieces of 1.5 to 2 mm and transferred onto an agar FNL medium containing the selection agent.

### Example 3

### Selection of the Soybean Transplastomic Lines

3.1. Evaluation of Soybean Sensitivity to Spectinomycin

Currently, only the aadA gene which confers resistance to spectinomycin has been used successfully as a marker for selection of transplastomic events. We initially verified the sensitivity of soybean to spectinomycin. In fact, some plant species such as rice are naturally resistant to spectinomycin since they have a mutated 16SrRNA. From this viewpoint, embryogenic soybean calli were placed on FNL medium supplemented with spectinomycin at a concentration of 100 mg/l, 300 mg/l (dose used in the prior art for selecting potato—Sidorov et al., 1999-), 500 mg/l (dose used in the prior art for selecting tobacco—Svab and Maliga, 1990; Svab et al., 1993), 600 mg/l and 700 mg/l. These calli were subcultured on the same medium after three weeks. For all these concentrations, the tissues begin to bleach after approximately two weeks, which shows the natural sensitivity of soybean to spectinomycin.

3.2. Selection of Transplastomic Lines

After 2 days on FNL medium, the embryogenic soybean calli bombarded with pCLT312 (as described above) are recovered and then transferred onto a sterile screening gauze so as to be in direct contact with an agar FNL selection medium containing 200 mg/l of spectinomycin. The tissues are subcultured on this same medium after 15 days, and then, after a further 15 days, on an agar FNL medium containing 300 mg/l of spectinomycin. After 20 days, they are again subcultured on the latter medium. According to this method of selection, only the transformed tissues remain green. The first green calli, which are resistant to spectinomycin, appear after 1.5 to 2 months. These putative plastid transformants are then maintained on an FNL medium supplemented with 150 mg/l of spectinomycin.

Eleven events resistant to spectinomycin (200 mg/l) were obtained from 4 bombardments (15 calli on average per bombardment). The first putative transformants appeared after 63 days (2 months). These calli were then amplified in liquid SBP6 medium (containing 150 mg/l of spectinomycin) so as to allow regeneration of plants and molecular analyses. The SBP6 medium is described in Finer and Nagasawa (1988, Development of an embryogenic suspension culture of soybean (Glycine max Merill.) Plant Cell. Tissue and Organ Culture 15: 125-136). It contains the following ingredients (in mg/l): Na₂EDTA: 37.24; FeSO₄.7H₂O: 27.84; MgSO₄.7H₂O: 370; MnSO₄.H₂O: 16.9; ZnSO₄.H₂O: 8.6; CuSO₄.7H₂O: 0.025; CaCl₂.2H₂O: 440; KI; 0.83; CoCl₂.6H₂O: 0.025; KH₂PO₄: 170; H₃BO₃: 6.2; Na₂MoO₄.2H₂O: 0.25; myoinositol: 100; nicotinic acid: 1; pyridoxine-HCl: 1; thiamine-HCl: 10; NH₄NO₃: 800; KNO₃: 3000; asparagine: 670; 6% sucrose; 2.4-D: 5; pH 5.7.

### Example 4

### Identification of the Soybean Transplastomic Lines and Study of the Homoplasmic State of these Various Lines by Southern Blotting

The transplastomic lines were identified by Southern blotting (Sambrook et al., 1989, Molecular Cloning: A Laboratory Manual, Nolan C. ed., New York: Cold Spring Harbor Laboratory Press) on calli and then on the plants derived from these calli.

The total DNA from 10 calli of the 11 spectinomycin-resistant calli were extracted with a commercial kit (Qiagen: “Dneasy Plant Mini Kit”). However, any DNA extraction technique known to those skilled in the art may be validly used (Sambrook et al., 1989, Molecular Cloning: A Laboratory Manual, Nolan C. ed., New York: Cold Spring Harbor Laboratory Press). One μg of DNA extracted from each of these 10 calli was then digested with the EcoRI restriction enzyme (Biolabs). This digestion makes it possible to generate fragments of interest with a size which can be exploited by Southern blotting, in particular a 4042 bp fragment for the transformed plastomes, a 2667 bp fragment for the wild-type plastomes, and a 2452 bp fragment for the transformed plastomes which have undergone a recombination between the two PrrnCs (tobacco and soybean). In fact, since the recombination mechanisms within the plastid are very active, the occurrence of a recombination between these two highly homologous sequence elements, oriented in the same direction, is possible.

The DNA fragments are separated by electrophoresis with slow migration overnight at 55V in a 0.8% agarose gel (QA Agarose™ Multipurpose, QBIOGENE). The transfer was then carried out conventionally (Maniatis et al., 1989). These DNA fragments are revealed by hybridization with radioactive (³²P-labeled) probes which are of two types: a probe which hybridizes to the aadA transgene (probe which reveals only the transplastomes) and a probe which hybridizes to a portion of the intergenic region of the plastid DNA (probe for visualizing the 3 plastome forms, corresponding to nucleotides 2293 to 3068 of the Glycine max plastome; Genebank X07675). These two probes were amplified by PCR (with the pair OSSG5—SEQ ID No. 6—and OSSG310—SEQ ID No. 7—for the probe which hybridizes to the intergenic region of the plastid DNA, and the pair OAAX3—SEQ ID No. 8—and OAAN5—SEQ ID No. 9—for the aadA probe), and then labeled with ³²p (Megaprime kit, AMERSHAM). The two membranes were washed with solutions of increasing stringency (6×SSC, then 2×SSC−0.1% SDS, and 0.1×SSC−0.1% SDS at 65° C.). After two hours of exposure at −80° C., with an intensifying screen, the autoradiogram revealed the presence of an expected band of 4042 pb (corresponding to the plastome transformed with aadA) in each of the 10 spectinomycin-resistant calli tested. All the spectinomycin-tolerant soybean events tested are therefore transplastomic. Unlike the plastid transformation of all the species obtained to date (Svab et al., 1993; Staub et al., 1993; Sidorov et al., 1999; Sikdar S. R. et al., 1998), no spontaneous mutant resistant to this antibiotic, due to specific mutations in the 16SrRNA plastid gene, was observed in our soybean transformation experiments.

Furthermore, nine of the ten events are in the homoplasmic state (or at least very close) since only callus number 1 still has copies of wild-type plastomes visible by Southern blotting. No recombination event between the two consecutive Prrns (tobacco PrrnC and native soybean Prrn), oriented in the same direction, was detected by this analysis.

### Example 5

### Regeneration of the Soybean Transplastomic Plants

The soybean transplastomic plants were regenerated in the following way. When sufficient tissues have been produced in FNL medium, they are then converted to embryos using a medium described by Finer and McMullen, in: Transformation of soybean via particle bombardment of embryogenic suspension culture tissue. In Vitro Cell. Dev. Biol. 27P: 175-182, 1991. After 3 to 4 transfers on this medium containing 150 mg/l of spectinomycin, the embryos are air-dried in a Petri dish for 2 days before germination on a Murashige and Skoog medium (vitamins B5) at half ionic strength (50% of the amounts of MS medium) with 15 g/l of saccharose, 150 mg/l of spectinomycin and 7 g/l of phytagar, pH 5.7. When the young plants are well developed (3-leaflet stage) and rooted, they are then transferred into a “jiffy pot” peat-based substrate for a period of 10-15 days for an acclimatriation phase before being transferred into a greenhouse. The plants are then grown in a greenhouse with culture conditions identical to those for non-transplastomic soybean. During flowering, the pollen is removed so as to perform artificial pollinization of the nontransgenic plants in order to verify the non-transmission of the spectinomycin resistance characteristic by these reproductive organs.

Furthermore, a control for correct transmission of the expression cassette and for the homoplasmic state of the descendants is carried out by PCR and Southern blotting. The seeds derived from the various transplastomic lines were sown on a medium of the Murashige and Skoog type at half ionic strength containing 15 g/l of saccharose and 500 mg/l of spectinomycin. All the seeds germinated and produced spectinomycin-tolerant plants, unlike wild-type seeds. This experiment thus demonstrates the stability and transmission of the expression cassette to the descendants. In addition, all the soybean transplastomic plants obtained are fertile. This is therefore the first report describing the production of a fertile transplastomic plant other than tobacco and tomato (Ruf et al., 2001). In fact, firstly, all the transplastomic events of A. thaliana and of rice produced to date were sterile (Sikdar et al., 1998; Khan and Maliga, 1999), and, secondly, it had never been possible to regenerate transformed soybean cells into fertile plants (Zhang et al., 2001).

### Example 6

### Expression of the 2maroA Gene in Soybean Plastids

6.1. Vector Construction

pCLT317, pCLT318, pCLT319 and pCLT320 vectors for the introduction of the double mutated aroA gene (2maroA) sequence between the trnV and rps12/7 genes in the inverted-repeat region of the Glycine max plastid genome derive from pCLT312 (as described in the example 1). All contain two adjacent and heterologous expression cassettes flanked by the LHRR and RHRR plastid sequences of soybean, identical to those of pCLT312. These two expression cassettes are in the same transcriptional orientation as the native soybean 16SrDNA gene (RRHR) in the plasmid pCLT318 and pCLT320 or in the inverted transcriptional orientation in the plasmid pCLT317 and pCLT319.

The selection cassette AADA contains the coding sequence of the aada gene transcribed from a synthetic promoter consisting of the promoter of the tobacco 16SrDNA gene (PrrnC) fused with the 5′ untranslated region of the tobacco plastid rbcL gene (5′rbcLNt), as described by Svab and Maliga (1993) and in the U.S. Pat. No. 5,877,402. The 3′psbA regulatory region was used to stabilize the mRNA of the gene of interest (Svab and Maliga, 1993; U.S. Pat. No. 5,877,402). The NotI-EcoRV fragment AADA was cloned in NotI/NruI restriction sites of pCLT405 (corresponding to the pMCS5 vector from Mobitech disrupted in the NcoI and XbaI restriction sites) to form the pCLT165. The XbaI restriction site present after the stop codon of the coding sequence of aadA was then eliminated in pCLT165 to give pCLT166 (containing the AADA-166 cassette; SEQ ID NO: 11).

The expression cassette of the 2maroA gene contains the plastid and nuclear encoded polymerase (PEP/NEP) promoters from the tobacco 16SrDNA gene (PrnnL), a ribosome-binding site (RBS) from the G10L (Ye et al., 2001, The Plant J. 25: 261-270; Hajdukiewicz, WO 01/04327), the 2maroA coding sequence (Stalker et al, 1985, J. Biol. Chem. 260(8): 4724-4728; AroA gene from Salmonella typhimurium containing two mutations introducing one Isoleucine at position 97 and one Serine at position 101) and the 3′ untranslated region of the tobacco plastid rbcL gene. In addition, in pCLT317 and pCLT318 plastid transformation vectors, the gene of interest is fused at its 5′ end (NcoI site) to the first 14 amino acids of the GFP protein (Ye et al., 2001, The Plant J. 25: 261-270; Pang et al.,1996, Plant Physiol. 112(3): 893-900) in order to enhance the translation efficiency or increase fusion protein stability.

The expression cassette was assembled from PCR-amplified plastid regulatory elements. The 16S rRNA promoter, PrnnL was amplified by PCR from total DNA of Nicotiana Tabacum (cv PBD6) using two specific primers:

otprrnc5:
5′-caattgtcgcgagaattcgctagcggcgccgctcccccgccgtcgtt
c-3′
and
otprrnc3:
5′-atcgatccgcgggagctcggtaccatgcatcgtctagattcggaatt
gtctttccttcc-3′.

The PCR fragment was cloned into the pPCRscript to form pCLT160. In order to eliminate potential ATG start codons, a C was inserted at the position 102, a G was deleted at the position 126, the A at the position 111 was converted to T and the T to G at the position 134. The resulting vector is called pCLT 161.

To synthesize the fusion of the 5′UTR from the G10L gene with the first 14 amino acids of the GFP (Pang et al., 1996) (G10L::14aaGFP), the following primers:

Og10L5:
5′-tatctagaaataattttgtttaactttaagaaggagatatacccatg
ggcaagggcg-3′,
and
Opgfp3:
5′-ggatgcattgcttaagattgggaccacgccagtgaacagttcctcgc
ccttgcccatgggtatatct-3′

were annealed to each other and elongated using standard PCR technology and Pwo DNA polymerase (Roche). These oligonucleotides were also engineered in order to create a XbaI restriction site at the 5′ end and BfrI and NsiI at the 3′ end of the fusion G10L::14aaGFP. A NcoI restriction site is inserted at the junction between the 5′UTR of the G10L gene and the 14aa of the GFP. This NcoI site offers the possibility to eliminate the 14aaGFP if necessary. The PCR fragment was cloned in the TOPO vector (Invitrogen) to form pCLT411.

The 2maroA gene from Salmonella typhimirium was amplified by PCR using oligonucleotides:

OaroAdb5:
5′-gccttaagctccatggaatccctgacgttacaaccc-3′,
and
OaroAdb3:
5′-gcgatgcataatttaaattaggcaggcgtactcattcg-3′.

A PCR fragment was purified and cloned in the pPCRscript vector (Stratagene) to yield pCLT406.

The 3′ untranslated region of the tobacco plastid rbcL gene (3′rbcLNt) (nucleotides 59,035 to 59,246 on the N. tabacum plastome; Shinozaki et al., 1986) was amplified by PCR from total DNA of Nicotiana Tabacum (cv PBD6) and cloned into the pPCRscript to form pCLT162. A DraIII/SwaI fragment containing the 3′rbcLNt was cloned downstream the 2maroA gene into the DraIII/SwaI sites of pCLT406 to form pCLT164. The 1517 bp BfrI/NsiI pCLT164 fragment carrying 2maroA::3′rbcLNt was cloned into pCLT411 opened with BfrI and NsiI restriction enzymes to yield pCLT169. The NsiI/XbaI G10L::14aaGFP::2maroA::3′rbcLNt fragment was cloned downstream the PrrnLNt into the pCLT161 to yield pCLT170 containing the complete AROA cassette (AROA-170; SEQ ID NO: 12). The NheI/NsiI AROA-170 cassette was cloned downstream the selection cassette AADA-166 into pCLT166 to form pCLT171.

The two expression cassettes AADA-166 and AROA-170 were further cloned between the two recombination regions RHRR and LHRR, identical to pCLT312 either in the same or in the inverse transcriptional orientation as the native soybean 16SrDNA gene (in RRHR). In order to create appropriate restriction sites for cloning, two multiple restriction sites (SMC1 and SMC2) were obtained using standard PCR technology by annealing and elongating the following oligonucleotides OSMC5 (5′-gaaagcttcggaccgtagtttaaacaggcccatatggcct-3′) with OSMC3 (5′-gactcgagttaattaatcggcgcgccaggccatatg-3′) for SMC1 and OSMC51 (5′-gagcggccgcctcgagcggaccgtagtttaaacaggcccatatggcct-3′) with OSMC31 (5′-gaaagcttttaattaatcggcgcgccaggccatatg-3′) for SMC2. The SMC1 and SMC2 were digested by HindIII and XhoI restriction enzyme and cloned into pCLT312 digested by the same enzymes to give respectively pCLT316 and pCLT315. The two expression cassettes AADA-166 and AROA-170 were cloned as a 3189 bp PmeI-PacI pCLT171 fragment into the PmeI and PacI restriction sites of pCLT315 and pCLT316 to form the plastid transformation vectors pCLT317 and pCLT318, respectively. In order to evaluate the influence of the 14aaGFP on expression of the transgene, pCLT317 and pCLT318 were digested by NcoI restriction enzyme to remove the 14aaGFP and ligated to yield pCLT319 and pCLT320, respectively. The expression cassettes of the 2maroA gene present in pCLT319 and pCLT320 are identical and are named AROA-319 (SEQ ID NO: 13). The expression cassettes are in the same transcriptional orientation as the native soybean 16SrDNA gene (RRHR) in the plasmids pCLT318 and pCLT320 or in the inverted transcriptional orientation in the plasmids pCLT317 and pCLT319.

All plastid transformation vectors were constructed in order to lead to an excision of the aadA gene after the integration of the cassettes inside the plastome. Indeed, the two transgenes are driven by a tobacco Pr -m present in the same transcriptional sense. The AADA-166 cassette being upstream the one of the gene of interest, an elimination of the selectable marker could be obtained by a homologous recombination between the two promoters.

6.2. Transformation

Plastid transformation experiments were carried out as described in the example 2 and 3 by bombardment of soybean embryogenic tissue, using gold particles coated with all the above-described plastid transformation vectors. Putative transformants were selected as described in the example 3 on spectinomycin medium. In order to distinguish transplastomic event from spontaneous mutant or nuclear transformant, PCR analysis were performed on total DNA from each antibiotic resistant callus obtained using several specific couple of oligonucleotides.

### Example 7

### Expression of the Heliomicin Gene in Soybean Plastids

7.1. Vector Construction

pCLT321 is derived from pCLT317. The NcoI/Blunt PCR heliomicin fragment amplified by PCR using the oligonucleotides P2 (5′-ACACCATGGATAAATTAATTGG-3′) and P3 (5′-CCTCTAGATTAAGTTTCACACCAAC-3′) from Heliothis virescens genome (WO 99/53053), and recoded for expression into tobacco plastids was cloned into the NcoI and SwaI restriction sites of pCLT317, replacing the 2maroA gene. pCLT321 carries the AADA-166 and the heliomicin (HELIO-321; SEQ ID NO: 14) cassettes in the inverse transcriptional orientation as the native soybean 16SrDNA gene. The HELIO-321 cassette is driven by the PrnnL fused with the RBS from the G10L but without the first 14aa of the GFP.

7.2. Transformation

Plastid transformation experiments were carried out as described in the example 2 and 3 by bombardment of soybean embryogenic tissue, using gold particles coated with all above-described plastid transformation vectors. Putative transformants were selected as described in the example 3 on spectinomycin medium. In order to distinguish transplastomic event from spontaneous mutant or nuclear transformant, PCR analysis were performed on total DNA from each antibiotic resistant callus obtained using several specific couple of oligonucleotides.

7.3. Analysis of Antifungal Transplastonic Soybean

The strategy for the PCR analysis of the transformants with pCLT321 was to land the primer P6 (5′-GTTAAGGTAACGACTTCGGCATGG-3′) immediately outside the RHRR in the soybean 16SrDNA gene, outside the homologous recombination region, while landing the other one P5 (5′-ctcagtactcgagttatttgccgactaccttggtgatctcgcc-3′) on the aadA gene. A 2,838 bp PCR product should be obtained in the case of integration of transgene into the plastome. The expected product was observed for the transgenic calli 1, 3, and 4 obtained using the soybean vector pCLT321. Unbombarded plants (controls) did not yield any PCR products, as expected. These PCR results show that the aadA gene is really integrated into the soybean plastome at the expected locus. The integration of the two expression cassettes into the soybean plastome was demonstrated using the primers P7 (5′-CATGGGTTCTGGCAATGCAATGTG-3′)/P8 (5′-CAGGATCGAACTCTCCATGAGATTCC-3′) designed to land on both sides of the site of integration of the foreign gene into the LHRR and RHHR, respectively. Two 1030 bp and 3054 bp PCR products should be observed for the WT plastome and the transplastome, respectively. The expected products were obtained for the WT and the transplastomic lines 1, 3 and 4. The spectinomycin resistant lines 1, 3 and 4 are thus transplastomic. The presence of some WT fragments indicated some heteroplasmy. An additional 1666 bp PCR fragment is observed in these three transplastomic lines corresponding probably to the recombined transplastome after excision of the AADA-166 cassette by homologous recombination. The integration of the two expression cassettes into the soybean plastome was confirmed using two other sets of primers P1 (5′-CGTATCGAATAGAACATGCTTAG-3′; landing on the LHRR)/P2 (5′-ACACCATGGATAAATTAATTGG-3′; on the heliomicin gene) and P4 (5′-CGTCATACTTGAAGCTAGACAGGC-3′; landing on aadA)/P3 (5′-CCTCTAGATTAAGTTTCACACCAAC-3′; on the heliomicin gene). Expected PCR products of 520 bp and 922 bp corresponding to the transplastome were obtained for the transplastomic event 1, 3, and 4 using the primers P1/P2 and P4/P3, respectively.

PCR screening for transplastomic events showed that 3 out of 4 resistant clones integrate the transgenes like the aadA gene linked to the Heliomicin gene into the soybean plastome. These 3 transplastomic events were advanced to further steps of regeneration

To determine the accumulation of heliomicin, Western Blot analysis was performed on a single transplastomic line, the event number 1. Total soluble cellular protein was extracted from leaves of wild type soybean and from embryos of transplastomic soybean. Western Blot was probed with anti-heliomicin antibodies. A dilution series of purified Heliomicin standard was used to quantify the expression of the heliomicin. The Western Blot results show a very weak accumulation of Heliomicin protein in the transplastomic lines. One of the reason could be the formation of insoluble inclusion bodies or a degradation of the heliomicin due to a misfolding of disulfide bonds present in the protein.

### Example 8

### Expression of the hppd Gene in Soybean Plastids

8.1. Vector Construction

The hppd gene from Pseudomonas fluorescens (Rüetschi et al., Eur. J. Biochem., 205, 459-466, 1992, WO 96/38567) was amplified by PCR using oligonucleotides Ohppd5 (5′-gccttaagctccatggcagatctatacgaaaacccaatgggc-3′) and Ohppd3 (5′-gccatttaaattaatcggcggtcaatacaccacgacgcacctg-3′). A 1099 bp PCR fragment was purified and cloned in the pPCRscript vector to yield pCLT409. A NcoI/SwaI pCLT409 fragment containing the hppd gene was cloned into the NcoI and SwaI restriction sites of pCLT317, resulting in pCLT323. pCLT323 carries the AADA-166 and the hppd (HPPD-323, SEQ ID NO: 15) cassettes in the inverse transcriptional orientation as the native soybean 16SrDNA gene. The HPPD-323 cassette is driven by the PrnnL fused with the RBS from the G10L but without the first 14aa of the GFP.

8.2. Transformation

Plastid transformation experiments were carried out as described in the example 2 and 3 by bombardment of soybean embryogenic tissue, using gold particles coated with all above-described plastid transformation vectors. Putative transformants were selected as described in the example 3 on spectinomycin medium. In order to distinguish transplastomic event from spontaneous mutant or nuclear transformant, PCR analysis were performed on total DNA from each antibiotic resistant callus obtained using several specific couple of oligonucleotides.

8.3. Analysis of Herbicide Tolerant Transplastomic Soybean

PCR analysis using one primer landing on the native plastome, outside the homologous recombination region, while landing the other on the aadA or hppd genes showed that the spectinomycin resistant calli are transplastomic.

In order to detect HPPD accumulation in the pCLT323 transplastomic event, embryos were grown on FNL media containing 1 ppm DKN, the active molecule of the herbicide isoxaflutole. Results show that, after 25 days of culture, transplastomic embryos are tolerant to 1 ppm DKN unlike WT embryos grown in the same conditions.

### Example 9

### Expression of the Cry1Ab Gene in Soybean Plastids

9.1. Vector Constriction

The cry1Ab gene from Bacillus thuringiensis (Bt) (GeneBank X04698) coding for the Cry1Ab protoxin was amplified by PCR using oligonucleotides OcryWT5 (5′-gccttaagctccatggataacaatccgaacatcaatg-3′) and OcryWTL3 (5′-gccatttaaattattcctccataagaagtaattccacgctgtccacg-3′) from Bacillus thuringiensis (strain berliner 1715) genome. The 5′ part of the cry1Ab gene coding for the toxin was also amplified by PCR using the oligonucleotides OcryWT5 and OcryWTC3 (5′-gccatttaaattaatcatattctgcctcaaaggttacttctgccggaac-3′).

A 3490 and 1873 bp PCR fragment for the cry1Ab genes coding for the protoxin Cry1Ab and the toxin Cry1Ab, respectively, was purified and cloned in the pPCRscript vector to yield pCLT408 and pCLT407, respectively.

A NcoI/SwaI pCLT408 fragment containing the cry1Ab gene was cloned into the NcoI and SwaI restriction sites of pCLT317, resulting in pCLT327 containing the cassette CRYL327 (SEQ ID NO: 17). A NcoI/SwaI pCLT407 fragment containing the toxin cry1Ab gene was cloned into the NcoI and SwaI restriction sites of pCLT317, resulting in pCLT329 containing the cassette CRYS329 (SEQ ID NO: 18). pCLT327 and pCLT329 carry the AADA-166 and the CRYL327 or CRYS329 cassettes in the inverse transcriptional orientation as the native soybean 16SrDNA gene. The CRYL327 or CRYS329 cassettes are driven by the PrrnL::G10L but without the first 14aa of the GFP.

A NcoI/SfiI pCLT317 fragment containing the PrrnL::G10L::14aaGFP was ligated into pCLT327 digested by the NcoI and SfiI restriction enzymes to form pCLT325 containing the CRYL325 cassette (SEQ ID NO: 16). pCLT325 carries the two expression cassettes AADA-166 and CRYL325 in the inverse transcriptional orientation as the native Prrn. A NcoI/SwaI pCLT325 fragment containing the cry1Ab gene coding for the protoxin was cloned into the Ncol and SwaI restriction sites of pCLT318, resulting in pCLT322. pCLT322 carries the two expression cassettes AADA-166 and CRYL327 in the same transcriptional orientation as the native Prrn. The protoxin cry1Ab gene is driven by the PrrnL::G10L but without the first 14aa of the GFP.

A SwaI/SfiI pCLT325 fragment containing PrrnL::G10L::14aaGFP was ligated into pCLT318 digested by the SwaI and SfiI restriction enzymes to form pCLT324. pCLT324 carries the two expression cassettes AADA-166 and CRYL325 in the same transcriptional orientation as the native Prrn. The cry1Ab gene coding for the protoxin is driven by the PrrnL::G10L::14aaGFP.

9.2. Transformation

Plastid transformation experiments were carried out as described in the example 2 and 3 by bombardment of soybean embryogenic tissue, using gold particles coated with all above-described plastid transformation vectors. Putative transformants were selected as described in the example 3 on spectinomycin medium. In order to distinguish transplastomic event from spontaneous mutant or nuclear transformant, PCR analysis were performed on total DNA from each antibiotic resistant callus obtained using several specific couple of oligonucleotides.

9.3. Analysis of Insect Resistant Transplastomic Soybean

PCR analysis using one primer landing on the native plastome, outside the homologous recombination region, while landing the other on the aadA or cry1Ab genes showed that the spectinomycin resistant calli are transplastomic.

Using Bt Cry1Ab FlashKits (ABC BioKits), Cry1Ab protein accumulation in embryos of transplastomic and WT soybean was examined. Results show the apparition of a band (red sample line) for the pCLT327 transplastomic event and not for the WT. The pCLT327 transplastomic event thus express the Cry1Ab protein.

1. A fertile transplastomic leguminous plant.

2. The fertile transplastomic leguminous plant as claimed in claim 1, characterized in that it is soybean.

3. The fertile transplastomic leguminous plant as claimed in claim 1, characterized in that it comprises at least one expression cassette inserted into a plastome intergenic region.

4. The fertile transplastomic leguminous plant as claimed in claim 3, characterized in that said plastome intergenic region is located between the TrnV gene and the rps12/7 operon.

5. The fertile transplastomic leguminous plant as claimed in claim 3, characterized in that said expression cassette is inserted between the soybean plastome sequences corresponding to the identifiers SEQ ID No. 1 and SEQ ID No. 2.

6. The fertile transplastomic leguminous plant as claimed in claim 3, characterized in that said expression cassette comprises, functionally linked to one another, at least one promoter which is functional in plastids from plant cells, a sequence encoding a protein and a terminator which is functional in plastids from plant cells.

7. A transformation vector suitable for leguminous plant plastid transformation, characterized in that it comprises at least two sequences homologous with a zone of the plastome of the leguminous plant to be transformed, said homologous sequences bordering at least one expression cassette.

8. The vector as claimed in claim 7, characterized in that the two sequences homologous with a zone of the plastome of the leguminous plant to be transformed correspond to sequences which allow integration of the expression cassette into a plastome intergenic region.

9. The vector as claimed in claim 7, characterized in that said zone corresponds to the region of the ribosomal RNA operon of the plastome.

10. The vector as claimed in claim 9, characterized in that one of the two homologous sequences comprises the genes encoding 16S ribosomal RNA (16SrRNA) and the Valine transfer RNA (TrnV), and in that the other homologous sequence comprises the intergenic region located between the TrnV gene and the rps12/7 operon.

11. The vector as claimed in claim 10, characterized in that the homologous sequence comprising the genes encoding the 16S ribosomal RNA (16SrRNA) and the Valine transfer RNA (TrnV) is represented by the sequence identifier SEQ ID No. 1, and in that the homologous sequence comprising the intergenic region located between the TrnV gene and the rps12/7 operon is represented by the sequence identifier SEQ ID No. 2.

12. The vector as claimed in claim 10, characterized in that the homologous sequence comprising the genes encoding the 16S ribosomal RNA (16SrRNA) and the Valine transfer RNA (TrnV) is positioned 5′ of the expression cassette, and in that the homologous sequence comprising the intergenic region located between the TrnV gene and the rps12/7 operon is positioned 3′ of the expression cassette.

13. The vector as claimed in claim 7, characterized in that said homologous sequences border, in addition to an expression cassette comprising a sequence encoding a protein of interest, at least one other expression cassette comprising a sequence encoding a selection marker.

14. A method for obtaining fertile transplastomic leguminous plants, characterized in that it comprises the steps of:
(a) transforming embryogenic tissues obtained from immature embryos of leguminous plants with a vector suitable for plastid transformation,
(b) selecting the transformed tissues,
(c) regenerating fertile transplastomic plants from the transformed tissues.

15. The method as claimed in claim 14, characterized in that the method of transformation used is the “particle bombardment” method.

16. The method as claimed in claim 14, characterized in that the vector suitable for plastid transformation comprises at least two sequences homologous with a zone of the plastome of the leguminous plant to be transformed, said homologous sequences bordering at least one expression cassette.
